(19) United States
(12) Patent Application Publication
(10) Pub. No.: US 2005/0123507 A1
(43) Pub. Date: Jun. 9, 2005

(54) Formulations for coated microprojections having controlled solubility

(76) Inventors: Mahmoud Ameri, Fremont, CA (US); WeiQi Lin, Palo Alto, CA (US); Michel J. N. Cormier, Mountain View, CA (US); Yuh-Fun Maa, Millbrae, CA (US)

Correspondence Address: Ralph C. Francis;Francis Law Group, 1942 Embarcadero, Oakland, CA, 94606, US

(21) Appl. No.: 11/034,891

(22) Filed: Jan. 12, 2005

Related U.S. Application Data
(63) Continuation-in-part of application No. 10/880,702, filed on Jun. 29, 2004.
(60) Provisional application No. 60/484,930, filed on Jul. 2, 2003.
(60) Provisional application No. 60/484,020, filed on Jun. 30, 2003.

(51) Int. Cl. A61K 38/21; A61K 38/28; A61K 38/22; A61K 38/09; A61K 38/14; A61K 9/70

(52) U.S. Cl. 424/85.1; 424/400; 514/12; 514/3; 514/18; 424/85.2; 424/85.4; 424/85.7; 514/16; 514/8; 604/500

(57) ABSTRACT

The invention provides for a formulation for coating one or more microprojections using a non-volatile counterion to improve solubility of a biologically active agent. The invention also includes formulations having a volatile counterion to reduce the solubility of a portion of the biologically active agent.

### CROSS-REFERENCE TO RELATED APPLICATIONS

This application is a continuation-in-part of U.S. application Ser. No. 10/880,702, filed Jun. 29, 2004, which claims the benefit of U.S. Provisional Application No. 60/484,930, filed Jul. 2, 2003.

### FIELD OF THE PRESENT INVENTION

This invention relates to the transdermal delivery of biologically active agents. More particularly, the invention relates to delivery of the agent using stratum corneum-piercing microprojections having a coating of the agent that has controlled solubility characteristics.

### BACKGROUND OF THE INVENTION

Agents are most conventionally administered either orally or by injection. Unfortunately, many medicaments are completely ineffective or have radically reduced efficacy when orally administered since they either are not absorbed or are adversely affected before entering the bloodstream and thus do not possess the desired activity. On the other hand, the direct injection of the medicament into the bloodstream, while assuring no modification of the medicament during administration, is a difficult, inconvenient, painful and uncomfortable procedure, sometimes resulting in poor patient compliance.

Transdermal delivery offers advantages over these conventional routes of administration. The word “transdermal” refers to delivery of a biologically active agent (e.g., a therapeutic agent, such as a drug) through the skin to the local tissue or systemic circulatory system without substantial cutting or piercing of the skin, such as cutting with a surgical knife or piercing the skin with a hypodermic needle. Transdermal delivery, when compared to oral delivery, avoids the harsh environment of the digestive tract, bypasses gastrointestinal metabolism, reduces first-pass effects, avoids the possible deactivation by digestive and liver enzymes and does not subject the digestive tract to the agent. Transdermal agent delivery also eliminates the associated pain and reduces the possibility of infection.

Despite these benefits, transdermal delivery of a biologically active agent presents certain challenges. For example, passive transdermal systems typically include a reservoir containing a high concentration of agent adapted to contact the skin to permit diffusion through the skin and into the body tissues or bloodstream of a patient.

The transdermal flux is also dependent upon the condition of the skin, the size and physical/chemical properties of the active agent, and the concentration gradient across the skin. In particular, the outermost skin layer, the stratum corneum, consists of flat, dead cells filled with keratin fibers (keratinocytes) surrounded by lipid bilayers. This highly-ordered structure of the lipid bilayers confers a relatively impermeable character to the stratum corneum. Because of this low permeability of the skin to many agents, passive transdermal delivery has had limited applications.

To overcome the barrier presented by the stratum corneum, there have been many attempts to mechanically penetrate or disrupt the outermost skin layers thereby creating pathways into the skin in order to enhance the amount of agent being transdermally delivered. For example, early vaccination devices, generally known as scarifiers, included a plurality of tines or needles that were applied to the skin to scratch or make small cuts in the area of application. However, such devices did not offer enough control over the delivery amount or rate of agent delivery.

Other devices that use tiny skin piercing elements to enhance transdermal agent delivery are disclosed in European Patent EP 0407063A1, U.S. Pat. Nos. 5,879,326, 3,814,097, 5,279,544, 5,250,023, 3,964,482, and Re. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; which are hereby incorporated in their entirety by reference. The piercing elements in some of these devices are extremely small; some having dimensions (i.e., a microblade length and width) of only about 25-400 μm and a microblade thickness of only about 5-50 μm. These tiny piercing/cutting elements make correspondingly small microslits/microcuts in the stratum corneum for enhanced transdermal agent delivery therethrough.

Generally, the noted systems include a reservoir for holding the agent and also a delivery system to transfer the agent from the reservoir through the stratum corneum, such as by hollow tines of the device itself. Alternatively, a coating containing the active agent can be deposited on the microprojections themselves. Such an approach is disclosed in published U.S. Patent Application Nos. 2002/0132054, 2002/0193729, 2002/0177839, and 2002/0128599; which are hereby incorporated in their entirety by reference.

Using a microprojection device to transdermally deliver an agent coated on the microprojections confers a number of benefits. However, the use of a coated microprojection generally provides only a bolus delivery. Also, it can be difficult to provide a coating formulation that is readily solubilized upon piercing the skin.

It is therefore an object of the present invention to provide a formulation for coating a transdermal microprojection delivery device that has controlled solubility when dried.

It is another object of the present invention is to provide a coating for a transdermal microprojection delivery device that, when dried, rapidly establishes a therapeutically relevant blood level of the agent when the delivery device is applied to a patient.

It is yet another object of the present invention to provide a coating for a transdermal microprojection delivery device that maintains a therapeutically relevant blood level of the agent in the patient for a desired period of time after application of the delivery device.

### SUMMARY OF THE INVENTION

In accordance with the above objects and those that will be mentioned and will become apparent below, the composition, device and method for transdermally delivering a biologically active agent in accordance with this invention generally comprises a formulation of a biologically active agent and a non-volatile counterion, wherein the non-volatile counterion causes the formation of a first species of the biologically active agent that has improved solubility when the formulation is dried. The first species of the agent dissolves quickly to provide rapid attainment of a therapeutically relevant blood level of the agent. The compositions of the invention are adapted for coating a transdermal delivery device having stratum corneum-piercing microprojections.

In a preferred embodiment of the invention, the formulation further includes a counterion comprising a volatile counterion, wherein the volatile counterion causes the formation of a second species of the biologically active agent that has reduced solubility when the formulation is dried. Thus, the second species of the agent dissolves at a slower rate to provide sustained blood levels of the agent.

Preferably, the non-volatile counterions of the invention include weak acids and weak bases, acidic zwitterions and basic zwitterions, and strong acids and strong bases. Also preferably, volatile counterions of the invention include weak acids or weak bases.

In one aspect of the invention, the addition of a non-volatile counterion results in the formation of a species of the biologically active agent that has improved solubility. In another aspect of the invention, the addition of a volatile counterion results in the formation of a species of the biologically active agent that has reduced solubility. In a preferred embodiment, the non-volatile counterion and the volatile counterion are added in approximately equimolar amounts.

In one embodiment of the invention, the non-volatile counterion comprises a non-volatile weak acid that presents at least one acidic pKa and a melting point higher than about 50° C. or a boiling point higher than about 170° C. at Pₐₜₘ. Preferably, such acids include citric acid, succinic acid, glycolic acid, gluconic acid, glucuronic acid, lactic acid, malic acid, pyruvic acid, tartaric acid, tartronic acid, and fumaric acid.

In an alternate embodiment of the invention, the non-volatile counterion comprises a non-volatile weak base that presents at least one basic pKa and a melting point higher than about 50° C. or a boiling point higher than about 170° C. at Pₐₜₘ. Preferably, such bases include monoethanolomine, diethanolamine, triethanolamine, tromethamine, methylglucamine, glucosamine.

In another embodiment of the invention, the non-volatile counterion comprises a non-volatile acidic zwitterion that presents at least two acidic pKa, and at least one basic pKa, so that there is at least one extra acidic group as compared to the number of basic groups. Preferably, such compounds include glutamic acid and aspartic acid. In an alternate embodiment of the invention, the non-volatile counterion comprises a non-volatile basic zwitterion that presents at least one acidic pKa, and at least two basic pKa's, so that there is at least one extra basic group as compared to the number of acidic groups. Preferably, such compounds include lysine, arginine, and histidine.

In yet another embodiment, the non-volatile counterion comprises a non-volatile strong acid that presents at least one pKa lower than about 2. Preferably, such acids include hydrochloric acid, hydrobromic acid, nitric acid, sulfonic acid, sulfuric acid, maleic acid, phosphoric acid, benzene sulfonic acid and methane sulfonic acid. In an alternate embodiment of the invention, the non-volatile counterion comprises a non-volatile strong base that presents at least one pKa higher than about 12. Preferably, such bases include sodium hydroxide, potassium hydroxide, calcium hydroxide, and magnesium hydroxide.

In a further embodiment of the invention, the volatile counterion comprises a weak acid that presents at least one pKa higher than about 2 and a melting point lower than about 50° C. or a boiling point lower than about 170° C. at Pₐₜₘ. Preferably, such acids include acetic acid, propionic acid, pentanoic acid and the like. In an alternate embodiment of the invention, the volatile counterion comprises a weak base that presents at least one pKa lower than about 12 and a melting point lower than about 50° C. or a boiling point lower than about 170° C. at Pₐₜₘ. Preferably, such bases include ammonia and morpholine.

In the noted embodiments, the volatile and non-volatile counterions are preferably present in amounts necessary to neutralize the charge present on the agent at the pH of the formulation. Excess of counterion (as the free acid or base or as a salt) can be added to the agent in order to control pH and to provide adequate buffering capacity.

In one aspect of the invention, the biologically active agent includes therapeutic agents in all the major therapeutic areas including, but not limited to, anti-infectives, such as antibiotics and antiviral agents; analgesics, including buprenorphine and analgesic combinations; anesthetics; anorexics; antiarthritics; antiasthmatic agents, such as terbutaline; anticonvulsants; antidepressants; antidiabetic agents; antidiarrheals; antihistamines; anti-inflammatory agents; antimigraine preparations; antimotion sickness preparations, such as scopolamine and ondansetron; antinauseants; antineoplastics; antiparkinsonism drugs; antipruritics; antipsychotics; antipyretics; antispasmodics, including gastrointestinal and urinary; anticholinergics; sympathomimetrics; xanthine derivatives; cardiovascular preparations, including calcium channel blockers such as nifedipine; beta blockers; beta-agonists, such as dobutamine and ritodrine; antiarrythmics; antihypertensives, such as atenolol; ACE inhibitors, such as ranitidine; diuretics; vasodilators, including general, coronary, peripheral, and cerebral; central nervous system stimulants; cough and cold preparations; decongestants; diagnostics; hormones, such as parathyroid hormone; hypnotics; immunosuppressants; muscle relaxants; parasympatholytics; parasympathomimetrics; prostaglandins; proteins; peptides; psychostimulants; sedatives; and tranquilizers. Other agents that can be added to the formulation in combination with the therapeutic agent include vasoconstrictors, anti healing agents, and pathway patency modulators.

In a preferred embodiment, the biologically active agent is selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones (e.g., HGH, HMG, desmopressin acetate, etc), follicle luteoids, aANF, growth factors, such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

In another preferred embodiment of the invention, the biologically active agent of the invention comprises a fentanyl-based agent. Preferably, the fentanyl-based agent includes, without limitation, fentanyl bases, fentanyl salts, simple derivatives of fentanyl and closely related molecules. Examples of pharmaceutically acceptable fentanyl salts formed in conjunction with the counterions of the invention include, without limitation, acetate, propionate, butyrate, pentanoate, hexanoate, heptanoate, levulinate, chloride, bromide, citrate, succinate, maleate, glycolate gluconate, glucuronate, 3-hydroxyisobutrate, 2 hydroxyisobutyrate, lactate, malate, pyruvate, fumarate, tartarate, tartronate, nitrte, phosphate, benzene sulfonate, methane sulfonate, sulfate, sulfonate, tricarballylicate, malonate, adipate, citraconate, glutarate, itaconate, mesaconate, citramalate, dimethylolpropionate, tiglicate, glycerate, methacrylate, isocrotonate, β-hydroxybutyrate, crotonate, angelate, hydracrylate, ascorbate, aspartate, glutamate. Examples of simple fentanyl derivatives include, without limitation, alpha-methyl fentanyl, 3-methyl fentanyl, and methyl fentanyl. Closely related molecules include, without limitation, remifentanyl, sufentanyl, alfentanyl, lofentanyl and carfentanyl.

In the noted embodiment, the non-volatile counterion is present in amounts necessary to neutralize the positive charge present on the fentanyl-based agent at the pH of the formulation. Excess of counterion (as the free acid or as a salt) can be added to the agent in order to control pH and to provide adequate buffering capacity. In the case of counterions bearing more than one negative charge, the fentanyl based agent can be added in excess of the acid. For example, the citrate salt of fentanyl can comprise the monocitrate or the hemicitrate.

In one embodiment of the invention, the coating formulation includes a fentanyl-based agent comprising in the range of approximately 1-60 wt. % of the coating formulation, more preferably, in the range of approximately 5-30 wt. % of the coating formulation.

Preferably, the pH of the coating formulation containing the fentanyl-based agent is below approximately pH 6. More preferably, the pH of the coating formulation is in the range of approximately pH 1-6. Even more preferably, the pH of the coating formulation is in the range of approximately pH 2-5.5.

In another embodiment of the invention, the coating formulation includes at least one buffer. Examples of suitable buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

In one embodiment of the invention, the coating formulation includes at least one antioxidant, which can comprise sequestering agents, such sodium citrate, citric acid, EDTA (ethylene-dinitrilo-tetraacetic acid) or a free radical scavenger, such as ascorbic acid, methionine, sodium ascorbate, and the like. Presently preferred antioxidants include EDTA and methionine.

In the noted embodiments of the invention, the concentration of the antioxidant is preferably in the range of approximately 0.01-20 wt. % of the coating formulation. More preferably, the concentration of the antioxidant is in the range of approximately 0.03-10 wt. % of the coating solution formulation.

In one embodiment of the invention, the coating formulation includes at least one surfactant, which can be zwitterionic, amphoteric, cationic, anionic, or nonionic. Suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols, such as laureth-4.

In the noted embodiments of the invention, the concentration of the surfactant is preferably in the range of approximately 0.01-20 wt. % of the coating formulation. More preferably, the concentration of the surfactant is in the range of approximately 0.05-1 wt. % of the coating solution formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropylcellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation.

In another embodiment, the coating formulation includes a hydrophilic polymer selected from the following group: hydroxyethyl starch, dextran, poly(vinyl alcohol), poly(ethylene oxide), poly(2-hydroxyethylmethacrylate), poly(n-vinyl pyrolidone), polyethylene glycol and like polymers and mixtures thereof.

In a preferred embodiment, the concentration of the hydrophilic polymer is in the range of approximately 1-30 wt. %, more preferably, in the range of approximately 1-20 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, a polysaccharide or a reducing sugar. Suitable non-reducing sugars include, for example, sucrose, trehalose, stachyose, or raffinose. Suitable polysaccharides include, for example, dextran, soluble starch, dextrin, and inulin. Suitable reducing sugars include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides, such as, for example, primeverose, vicianose, rutinose, scillabiose, cellobiose, gentiobiose, lactose, lactulose, maltose, melibiose, sophorose, and turanose, and the like.

Preferably, the concentration of the stabilizing agent in the coating formulation is at a ratio of approximately 0.1-2.0%, more preferably, at a ratio of approximately 0.25-1.0% with respect to the biologically active agent.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilizing/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred solubilizing/complexing agents are beta-cyclodextrin, hydroxypropyl beta-cyclodextrin, 2-hydroxypropyl-beta-Cyclodextrin and sulfobutylether7 beta-cyclodextrin.

The concentration of the solubilizing/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt. % to 50 wt. % of the coating formulation.

Preferably, the coating formulations have a viscosity less than approximately 500 centipoise and greater than 3 centipoise.

The invention also comprises transdermal delivery devices having at least one microprojection configured to pierce the stratum corneum, the microprojection being coated with a biocompatible coating formed from one of the aforementioned coating formulations.

In one embodiment of the invention, the thickness of the biocompatible coating is preferably less than approximately 25 microns, more preferably, less than approximately 10 microns.

In one embodiment of the invention, the delivery device has a microprojection density of at least approximately 10 microprojections/cm², more preferably, in the range of at least approximately 200-2000 microprojections/cm².

In yet another embodiment, the microprojection is constructed out of stainless steel, titanium, nickel titanium alloys, or similar biocompatible materials, such as polymeric materials.

In another embodiment, the microprojection is constructed out of a non-conductive material, such as a polymer. Alternatively, the microprojection can be coated with a non-conductive material, such as polyparaxylene, polymonochloroparaxylylene or polydichloroparaxylylene (Parylene®), or a hydrophobic material, such as polytetrafluoroethylene (Teflon®), silicon or other low energy material.

Generally, the methods of the invention for transdermally delivering a biologically active agent comprise the steps of providing a transdermal delivery device having at least one stratum corneum-piercing microprojection, the microprojection including a biocompatible coating comprising a dried formulation of the biologically active agent and a non-volatile counterion, wherein the non-volatile counterion causes the formation of a first species of the biologically active agent that has improved solubility when the formulation is dried, and applying the delivery device to a patient to deliver the biologically active agent.

In one embodiment of the invention, applying the delivery device to a patient rapidly establishes a therapeutically relevant blood level of the agent in the patient. In a preferred embodiment, a therapeutically relevant blood level of the agent is established in less than 30 min after applying the device. More preferably, the therapeutically relevant blood level of agent is established in less than 15 min after applying the device. In the noted embodiments, the agent preferably comprises a fentanyl-based agent.

In a further embodiment of the invention, the step of providing a transdermal delivery device comprises providing a transdermal delivery device having a biocompatible coating comprising a dried formulation of the biologically active agent, a non-volatile counterion, and a volatile counterion, wherein the non-volatile counterion causes the formation of a first species of the biologically active agent that has improved solubility when the formulation is dried and wherein the volatile counterion causes the formation of a second species of the biologically active agent that has reduced solubility when the formulation is dried.

In such an embodiment, the step of applying the device to a patient provides and maintains a therapeutically relevant blood level of the agent for a desired period of time. Preferably, the therapeutically relevant blood level is maintained for a period in the range of approximately 1 to 6 hours, and more preferably, in the range of approximately 2 to 4 hours. In the noted embodiments, a preferred agent comprises fentanyl.

In embodiments of the invention wherein the biologically active agent comprise fentanyl, the therapeutically relevant blood level is at least approximately 0.3 ng/mL. Also preferably, the total dose of the fentanyl-based agent delivered transdermally is in the range of approximately 0.01 to 1 mg per day.

Another embodiment of the invention comprises a method for applying a biocompatible coating to a transdermal delivery device that has a least one stratum corneum-piercing microprojection that includes the steps of providing a formulation of a biologically active agent and a non-volatile counterion, applying the formulation to the microprojection and drying the formulation to form the coating, wherein the non-volatile counterion causes the formation of a first species of the biologically active agent that has improved solubility when the formulation is dried.

In a further embodiment of the invention, the step of providing a formulation comprises providing a formulation of a biologically active agent, a non-volatile counterion, and a volatile counterion, wherein the non-volatile counterion causes the formation of a first species of the biologically active agent that has improved solubility when the formulation is dried and wherein the volatile counterion causes the formation of a second species of the biologically active agent that has reduced solubility when the formulation is dried.

### BRIEF DESCRIPTION OF THE DRAWINGS

The invention will now be described in greater detail with reference to the preferred embodiments illustrated in the accompanying drawings and figures wherein:

FIG. 1 is a graph showing the charge profile of acetic acid (pKa 4.75) as a function of pH;

FIG. 2 is a graph showing the mole ratios of uncharged acetic acid and charged acetate ion as a function of pH;

FIG. 3 is a graph showing the charge profile of fentanyl as a function of pH.

FIG. 4 is a graph showing the mole ratios of the neutral and charged fentanyl species as a function of pH;

FIG. 5 is a graph showing the charge profile of hPTH (1-34)OH as a function of pH;

FIG. 6 is a graph showing the mole ratios of the net charged species of hPTH as a function of pH;

FIG. 7 is a graph showing the mole ratios of fentanyl acetate, acetic acid and the neutral form of fentanyl as a function of pH;

FIG. 8 is a graph showing the mole ratios for acetic acid the neutral form of hPTH(1-34)OH as function of pH;

FIG. 9 is a graph showing the charge profile of a peptide which is a hGRF analog;

FIG. 10 is a diagram depicting the loss of volatile counterion from the outer layer of a coating;

FIG. 11 is a perspective view of a microprojection array that could be used in conjunction with the present invention; and

FIG. 12 is a perspective view of a microprojection array showing several microprojections that have been coated.

### DETAILED DESCRIPTION OF THE INVENTION

### Definitions

Unless stated otherwise the following terms used herein have the following meanings.

The term “transdermal” means the delivery of an agent into and/or through the skin for local or systemic therapy.

The term “transdermal flux” means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more agents may be coated onto the microprojections resulting in co-delivery of the agents.

The term “biologically active agent” or “active agent”, as used herein, refers to a composition of matter or mixture containing an agent which is pharmacologically effective when administered in a therapeutically effective amount.

Such biologically active agents include therapeutic agents in all the major therapeutic areas including, but not limited to, anti-infectives, such as antibiotics and antiviral agents; analgesics, including buprenorphine and analgesic combinations; anesthetics; anorexics; antiarthritics; antiasthmatic agents, such as terbutaline; anticonvulsants; antidepressants; antidiabetic agents; antidiarrheals; antihistamines; anti-inflammatory agents; antimigraine preparations; antimotion sickness preparations, such as scopolamine and ondansetron; antinauseants; antineoplastics; antiparkinsonism drugs; antipruritics; antipsychotics; antipyretics; antispasmodics, including gastrointestinal and urinary; anticholinergics; sympathomimetrics; xanthine derivatives; cardiovascular preparations, including calcium channel blockers, such as nifedipine; beta blockers; beta-agonists, such as dobutamine and ritodrine; antiarrythmics; antihypertensives, such as atenolol; ACE inhibitors, such as ranitidine; diuretics; vasodilators, including general, coronary, peripheral, and cerebral; central nervous system stimulants; cough and cold preparations; decongestants; diagnostics; hormones, such as parathyroid hormone; hypnotics; immunosuppressants; muscle relaxants; parasympatholytics; parasympathomimetrics; prostaglandins; proteins; peptides; psychostimulants; sedatives; and tranquilizers. Other suitable active agents include vasoconstrictors, anti-healing agents and pathway patency modulators.

Further specific examples of active agents include, without limitation, growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones (e.g., HGH, HMG, desmopressin acetate, etc), follicle luteoids, aANF, growth factors, such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, BNP, VEGF, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

Particularly preferred biologically active agents of the invention include fentanyl-based agents (or analgesics). Fentanyl-based agents include, without limitation, fentanyl bases, fentanyl salts, simple derivatives of fentanyl and closely related molecules. Examples of pharmaceutically acceptable fentanyl salts formed with the counterions of the invention include, without limitation, acetate, propionate, butyrate, pentanoate, hexanoate, heptanoate, levulinate, chloride, bromide, citrate, succinate, maleate, glycolate gluconate, glucuronate, 3-hydroxyisobutrate, 2-hydroxyisobutyrate, lactate, malate, pyruvate, fumarate, tartarate, tartronate, nitrte, phosphate, benzene sulfonate, methane sulfonate, sulfate, sulfonate, tricarballylicate, malonate, adipate, citraconate, glutarate, itaconate, mesaconate, citramalate, dimethylolpropionate, tiglicate, glycerate, methacrylate, isocrotonate, β-hydroxybutyrate, crotonate, angelate, hydracrylate, ascorbate, aspartate, glutamate. Examples of simple fentanyl derivatives include, without limitation, alpha-methyl fentanyl, 3-methyl fentanyl, and methyl fentanyl. Closely related molecules include, without limitation, remifentanyl, sufentanyl, alfentanyl, lofentanyl, and carfentanyl.

It is to be understood that more than one agent may be incorporated into the agent formulation in the method of this invention, and that the use of the term “active agent” in no way excludes the use of two or more such agents or drugs. The agents can be in various forms, such as free bases, acids, charged or uncharged molecules, components of molecular complexes or nonirritating, pharmacologically acceptable salts. Also, simple derivatives of the agents (such as ethers, esters, amides, etc) which are easily hydrolyzed at body pH, enzymes, etc., can be employed.

The terms “therapeutically relevant blood level,” “biologically effective amount” or “biologically effective rate” shall be used when the biologically active agent is a pharmaceutically active agent and refers to the amount or rate of the pharmacologically active agent needed to affect the desired therapeutic, often beneficial, result. The amount of agent employed in the coatings will be that amount necessary to deliver a therapeutically relevant amount of the agent to achieve the desired therapeutic result. In practice, this can vary significantly depending upon the particular pharmacologically active agent being delivered, the site of delivery, the severity of the condition being treated, the desired therapeutic effect and the dissolution and release kinetics for delivery of the agent from the coating into skin tissues. For these reasons, it is not practical to generically define a precise range for the therapeutically effective amount of the agents of the invention according to the methods described herein.

The term “microprojections” refers to piercing elements which are adapted to pierce or cut through the stratum corneum into the underlying epidermis layer, or epidermis and dermis layers, of the skin of a living animal, particularly a human. The piercing elements should not pierce the skin to a depth which causes bleeding. Typically the piercing elements have a blade length of less than 500 μm, and preferably less than 250 μm. The microprojections typically have a width and thickness in the range of approximately 5 to 50 μm. The microprojections may be formed in different shapes, such as needles, hollow needles, blades, pins, punches, and combinations thereof.

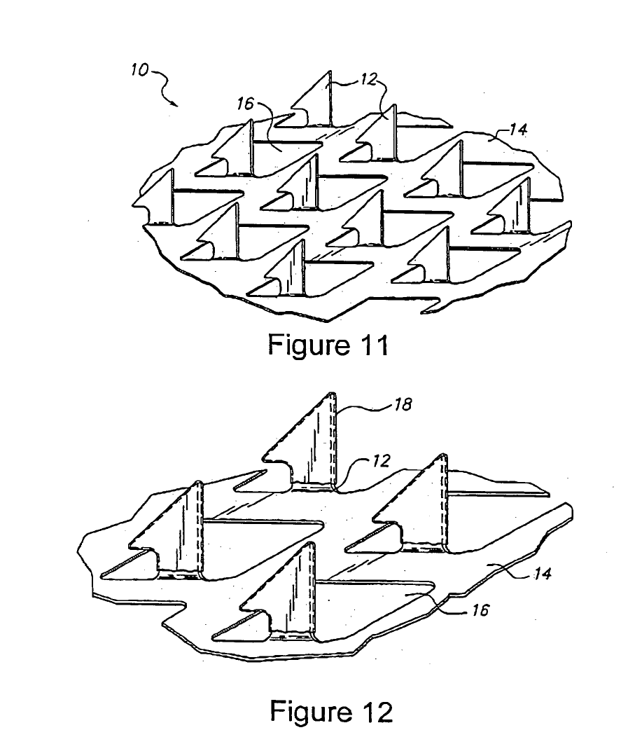

The terms “microprojection array” and “microprojection member” as used herein refers to a plurality of microprojections arranged in an array for piercing the stratum corneum to form a transdermal delivery device. The microprojection array may be formed by etching or punching a plurality of microprojections from a thin sheet and folding or bending the microprojections out of the plane of the sheet to form a configuration such as that shown in FIG. 11. The microprojection array may also be formed in other known manners, such as by forming one or more strips having microprojections along an edge of each of the strip(s) as disclosed in Zuck, U.S. Pat. No. 6,050,988.

The term “polyelectrolyte,” as used herein, means formulations of biologically active agents having ionic species. As is well known in the art, a polyelectrolyte is a macromolecular substance, which, on dissolving in water or another ionizing solvent, dissociates to provide multiply charged anions or cations. For example, agents comprising polypeptides frequently have complex ionic characters resulting from multiple amino acid residues having acidic and basic functionalities. The formulations can also include buffers or other adjuvants.

Volatile counterions are defined as weak acids presenting at least one pKa higher than about 2 and a melting point lower than about 50° C. or a boiling point lower than about 170° C. at Pₐₜₘ. Examples of such acids include acetic acid, propionic acid, pentanoic acid and the like. Volatile counterions are also defined as weak bases presenting at least one pKa lower than about 12 and a melting point lower than about 50° C. or a boiling point lower than about 170° C. at Pₐₜₘ. Examples of such bases include ammonia and morpholine.

Non-volatile counterions are defined as weak acids presenting at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid. Non-volatile counterions are also defined as acidic zwitterions presenting at least two acidic pKa, and at least one basic pKa, so that there is at least one extra acidic group as compared to the number of basic groups. Examples of such compounds include glutamic acid and aspartic acid.

Non-volatile counterions are also defined as weak bases presenting at least one basic pKa and a melting point higher than about 50° C. or a boiling point higher than about 170° C. at Pₐₜₘ. Examples of such bases include monoethanolomine, diethanolamine, triethanolamine, tromethamine, methylglucamine, glucosamine. Non-volatile counterions are also defined as basic zwitterions presenting at least one acidic pKa, and at least two basic pKa's, wherein the number of basic pKa's is greater than the number of acidic pkA's. Examples of such compounds include lysine, arginine, and histidine.

Non-volatile counterions are also defined as strong acids presenting at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid. Non-volatile counterions are further defined as strong bases presenting at least one pKa higher than about 12. Examples of such bases include sodium hydroxide, potassium hydroxide, calcium hydroxide, and magnesium hydroxide.

When referring to the volatility of a counterion, reference will always be made to the volatility of the non-ionized form of the counterion (e.g., acetic acid versus acetate).

Agents that behave like strong bases or strong acids (e.g., quaternary ammonium salts such as clidinium bromide or glycopyrrolate, sulfate derivatives, such as pentosan polysulfate, some phosphoric derivatives such as nucleic acids) generally are totally ionized in a wide range of pH (i.e. 4-10). The noted pH range covers conditions commonly used with pharmaceutical formulations.

Other compounds, such as neutral polysaccharides (e.g., inulin and dextrans), do not present acidic or basic functions. Since solubility in water is not significantly affected by pH for such classes of agents, they are generally not suitable for practicing the invention.

Conversely, many agents behave as weak acids or weak bases. Their neutral species usually present low water solubility. For example, the neutral species of many small molecular compounds, such as fentanyl, or peptides, such as PTH (1-34)OH, are notoriously insoluble in water. These compounds exhibit maximum solubility in water when they are in an electrically charged state. Because of their weakly acidic or basic nature, the respective concentrations of the neutral and ionized species, and therefore the solubility in water, is pH dependant. The invention applies to this class of agents.

Accordingly, the invention includes compositions of a biologically active agent with a non-volatile counterion sufficient to minimize the presence of the neutral form of the agent to assure enhanced solubility of the agent in the formulation, stability during storage in the solid state, and dissolution in the biological fluids at the time of administration.

Suitable biologically active agents of the invention present at least one weak acidic and/or one weak basic function and are present as a neutral species in the pH range 4 to 10. The mole ratio between the uncharged species and the charged species should be at least 1 to 100 in this pH range. Correspondingly, the volatile and non-volatile counterions are preferably present in amounts necessary to neutralize the charge present on the agent at the pH of the formulation. Excess of counterion (as the free acid or base or as a salt) can be added to the agent in order to control pH and to provide adequate buffering capacity.

The amount of non volatile counterion in the coating formulation should represent no more than 99%, preferably no more than 90%, of the amount necessary to neutralize the charge present on the agent at the pH of the formulation. The amount of volatile counterion should represent at least 1%, and preferably more than 10% of the amount necessary to neutralize the charge present on the agent at the pH of the coating formulation.

Following coating and drying, a substantial fraction of the volatile counterion is lost. This, in turn, results in formation of less charged and less water soluble species in the solid formulation.

The coating formulation preferably comprises water or another volatile solvent such as ethanol, isopropanol, methanol, benzene, acetone, ethyl ether, and the like, and mixture thereof.

Alternatively, a similar result can be achieved by mixing the non-volatile salt of the therapeutic agent with the net neutral species of the same agent. The amount of the non-volatile salt of the therapeutic agent should represent no more than 99%, preferably no more than 90%, of the molar fraction of the agent and the amount of net neutral species should represent at least 1%, and preferably more than 10%, of the molar fraction of the agent. The mixture is preferably solubilized or suspended in an adequate coating volatile solvent such as water, ethanol, isopropanol, methanol, benzene, acetone, ethyl ether, and the like, and mixture thereof.

In both cases, the charged species of the biologically active agent quickly dissolves when the microprojection member is applied to the patient, providing a bolus delivery that results in rapid elevation of the agent to therapeutically relevant blood levels. In turn, the reduced solubility species allows sustained delivery of the biologically active agent, providing delivery that maintains a therapeutically relevant blood level for a desired period of time.

In a presently preferred example, a fentanyl-based agent is formulated for transdermal delivery to provide “breakthrough pain” management. For “breakthrough pain” management, the preferred pharmacokinetic profile in humans includes establishment of therapeutically relevant blood levels in less than 30 min, preferably, less than 15 min. In addition, the therapeutically relevant blood levels should be sustained for at least 1 hour and up to 6 hours, preferably, 2-4 hours. In the case of fentanyl, the therapeutically relevant blood levels correspond to at least 0.3 ng/mL. Also, the total dose of the fentanyl-based agent delivered transdermally is preferably in the range of approximately 0.01 to 1 mg per day.

In one embodiment, the invention includes a formulation of volatile and non-volatile counterions with a fentanyl-based agent. For example, the fentanyl-based agent is mixed with an equimolar amount of the volatile counterion (e.g., acetic acid) and the non-volatile counterion (e.g., tartaric acid). Upon coating, some of the acetic acid will volatilize leaving a solid coating of fentanyl base on the microprojections and substantially no tartaric acid will volatilize leaving a solid coating of fentanyl tartarate on the microprojections. Upon administration into a patient, the fentanyl tartarate will exhibit improved solubility and promote the fast onset of action. Correspondingly, the fentanyl base will exhibit reduced solubility to yield a long lasting analgesia.

The solid coating is preferably obtained by drying a formulation on the microprojection as described in U.S. Patent Application Publication No. 2002/0128599. Other suitable processes can be employed, as described below. The formulation is usually an aqueous formulation. During the drying process, all volatiles, including water are mostly removed (the final solid coating still contains up to about 10% water). If a volatile compound that is in equilibrium between its ionized and non-ionized forms is present in solution, only the non-ionized form disappears from the formulation at the time where the drying process takes place and the ionized form stays in solution and incorporated into the coating.

As is known in the art, the kinetics of the agent-containing coating dissolution and release will depend on many factors including the nature of the agent, the coating process, the coating thickness and the coating composition (e.g., the presence of coating formulation additives). Depending on the release kinetics profile, it may be necessary to maintain the coated microprojections in piercing relation with the skin for extended periods of time (e.g., up to about 8 hours). This can be accomplished by anchoring the delivery device to the skin using adhesives or by using anchored microprojections such as described in WO 97/48440, incorporated by reference in its entirety.

Further embodiments of the present invention include a device having a plurality of stratum corneum-piercing microprojections extending therefrom. The microprojections are adapted to pierce through the stratum corneum into the underlying epidermis layer, or epidermis and dermis layers, but do not penetrate so deep as to reach the capillary beds and cause significant bleeding. The microprojections have a dry coating thereon which contains the biologically active agent. The coating is formulated to contain a non-volatile counterion to create an ionic species of the biologically active agent that has enhanced solubility upon piercing the skin. Additionally, the coating can contain a volatile counterion to create a species of the biologically active agent that has reduced solubility.

FIG. 11 illustrates one embodiment of a stratum corneum-piercing microprojection transdermal delivery device for use with the present invention. FIG. 11 shows a portion of the device having a plurality of microprojections 10. The microprojections 10 extend at substantially a 90° angle from a sheet 12 having openings 14. The sheet 12 may be incorporated in a delivery patch including a backing for the sheet 12 and may additionally include adhesive for adhering the patch to the skin.

In this embodiment the microprojections are formed by etching or punching a plurality of microprojections 10 from a thin metal sheet 12 and bending the microprojections 10 out of a plane of the sheet. Metals such as stainless steel and titanium are preferred. Metal microprojections are disclosed in Trautman et al, U.S. Pat. No. 6,083,196; Zuck, U.S. Pat. No. 6,050,988; and Daddona et al., U.S. Pat. No. 6,091,975; the disclosures of which are incorporated herein by reference.

Other microprojections that can be used with the present invention are formed by etching silicon using silicon chip etching techniques or by molding plastic using etched micro-molds. Silicon and plastic microprojections are disclosed in Godshall et al., U.S. Pat. No. 5,879,326; the disclosure of which is incorporated herein by reference.

FIG. 12 illustrates the microprojection transdermal delivery device having microprojections 10 having a biologically active agent-containing coating 16. The coating 16 may partially or completely cover the microprojection 10. For example, the coating can be in a dry pattern coating on the microprojections. The coatings can be applied before or after the microprojections are formed.

As discussed above, a number of other known methods can be employed to apply the coating to the microprojections. One such method is dip-coating, which can be described as a means to coat the microprojections by partially or totally immersing the microprojections into the agent-containing coating solution. Alternatively the entire device can be immersed into the coating solution. Coating only those portions the microprojection or microprojections that pierce the skin is preferred.

By use of the partial immersion technique described above, it is possible to limit the coating to only the tips of the microprojections. There is also a roller coating mechanism that limits the coating to the tips of the microprojection. This technique is described in a U.S. patent application Ser. No. 10/099,604, filed 15 Mar. 2002; which is fully incorporated herein by reference.

Other coating methods include spraying the coating solution onto the microprojections. Spraying can encompass formation of an aerosol suspension of the coating composition. In a preferred embodiment, an aerosol suspension forming a droplet size of about 10 to 200 picoliters is sprayed onto the microprojections and then dried. In another embodiment, a very small quantity of the coating solution can be deposited onto the microprojections as a pattern coating 18. The pattern coating 18 can be applied using a dispensing system for positioning the deposited liquid onto the microprojection surface. The quantity of the deposited liquid is preferably in the range of 0.5 to 20 nanoliters/microprojection. Examples of suitable precision metered liquid dispensers are disclosed in U.S. Pat. Nos. 5,916,524; 5,743,960; 5,741,554; and 5,738,728; the disclosures of which are incorporated herein by reference.

Microprojection coating solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

In all cases, after a coating has been applied, the coating solution is dried onto the microprojections by various means. In a preferred embodiment the coated device is dried in ambient room conditions. However, various temperatures and humidity levels can be used to dry the coating solution onto the microprojections. Additionally, the devices can be heated, lyophilized, freeze dried or similar techniques used to remove the water from the coating.

A number of factors affect the volatility of compounds. These include temperature, atmospheric pressure, and vapor pressure of the compound. The volatilization process is time dependant. In addition, ionized compounds present a much lower volatility as compared to their unionized forms. For example, acetic acid has a boiling point of 118° C. while sodium acetate is essentially non-volatile. If a volatile compound in equilibrium between its ionized and non-ionized forms is present in a solution, only the non-ionized form disappears from the solution and the ionized form stays in solution.

If the volatile compound is a weak acid, designated “AH”, the following equilibrium takes place in solution:
AH [symbol] A⁻+H⁺

With Ka1 being the equilibrium constant for AH, the equilibrium can be written as:
Ka1=(A⁻)×(H⁺)/(AH), where
(A⁻), (H⁺) and (AH) represent the concentrations of the species present in solution.

If AH is volatile, the equilibrium will shift towards A⁻+H⁺ [symbol] AH in order to satisfy the laws of equilibrium. Ultimately, the entire mass of the volatile weak acid will disappear from the solution.

If the volatile compound is a weak base (B) the following equilibrium takes place:
B+H⁺ [symbol] BH⁺

With Ka2 being the equilibrium constant, the equilibrium can be written as:
Ka2=(B)×(H⁺)/(BH⁺), where
(B), (H⁺), and (BH⁺) represent the concentrations of the species present in solution.

If B is volatile, the equilibrium will shift towards BH+ [symbol] B+H+ in order to satisfy the laws of equilibrium. As above, the entire mass of the volatile weak base will disappear from the solution.

When a weak acid and a weak base are mixed in solution, the acid and base form a salt according to the following equilibrium:
AH+B [symbol] A⁻+BH⁺

With Ka1 and Ka2 representing the equilibrium constants for AH and B, respectively, the equilibrium can be written as:
Ka1/Ka2=(A⁻)×(BH⁺)/(AH)×(B)

If AH is volatile, the equilibrium will shift towards A⁻+BH⁺ [symbol] AH+B in order to satisfy the laws of equilibrium. The net result will be an increase in the concentration of the free base and a resulting increase in pH. Conversely, if B is volatile, the equilibrium will shift identically with a net result of an increase in the concentration of the free acid and a decrease in pH.

Strong acids present a particular case because typically they are highly volatile. Indeed, hydrochloric acid is a gas in ambient conditions. When combined with a base, the strong acids form non-volatile salts because volatile strong acids are completely ionized in a wide pH range with the exception of extreme pH for some acids. In solution, or in the solid state, volatilization of the counterion occurs at the interface between the solution and the atmosphere or the solid and the atmosphere. In a solution, the high diffusivity of solutes minimizes differences in concentration between the interface and the bulk of the solution.

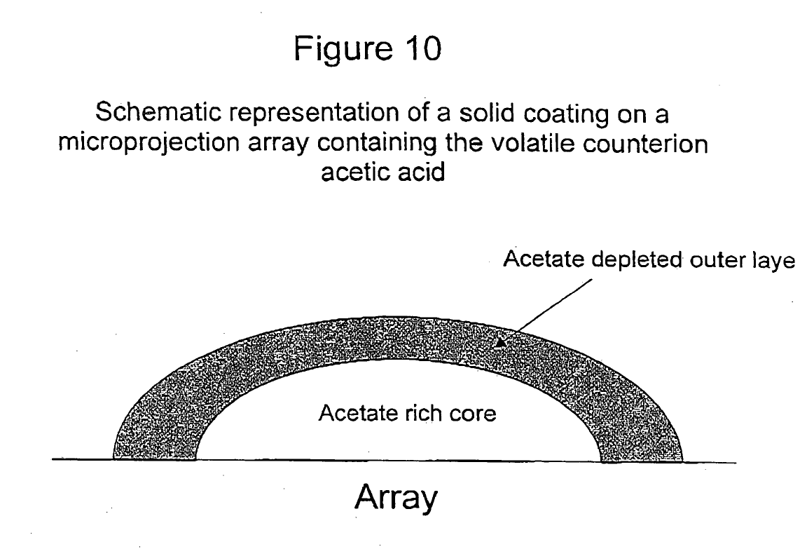

Conversely, in a solid state, diffusivity is very slow or non-existent and greater concentration gradients of the volatile counterion are achieved between the interface and the bulk of the solution. Ultimately, the outer layer of the coating is depleted in counterion while the bulk of the solid coating is relatively unchanged as compared to the initial dry state (see FIG. 10). This situation can produce a reduced solubility outer coating if the counterion is associated with an agent that is substantially insoluble in its neutral net charge state. Indeed, as will be explained in detail in Example 1, volatilization of the counterion results in formation of the reduced solubility neutral species. Thus, the volatilization reduces dissolution of the agent from the solid coating upon exposure to the biological fluids.

As is known in the art, specifics of the coating formulations depend upon the biologically active agent. For example, in certain embodiments of the invention, the biologically active agent comprises a fentanyl-based agent. In such embodiments, the acidic non-volatile counterion is present in amounts necessary to neutralize the positive charge present at the pH of the formulation. Excess of counterion (as the free acid or as a salt) can be added to the agent in order to control pH and to provide adequate buffering capacity. In embodiments including counterions that present more than one negative charge, the fentanyl-based agent can be added in excess of the acid. For example, the citrate salt of fentanyl can comprise the monocitrate or hemicitrate.

Further specific embodiments of the invention directed to the use of fentanyl-based agents include a coating formulation wherein the fentanyl-based agent is in the range of approximately 1-60 wt. % of the coating formulation, more preferably, in the range of approximately 5-30 wt. %. Also, preferably, the pH of the coating formulation containing a fentanyl-based agent is below approximately pH 6. More preferably, the pH of the coating formulation is in the range of approximately pH 1-6. Even more preferably, the pH of the coating formulation is in the range of approximately pH 2-5.5.

In other embodiments of the invention, known formulation adjuvants can be added to the coating solution as long as they do not adversely affect the necessary solubility and viscosity characteristics of the coating solution and the physical integrity of the dried coating.

To improve the therapeutic effect of the biologically active agent or to improve aspects of transdermal delivery, a number of additional compounds can be included in the formulations of the invention, as described below.

In another embodiment of the invention, the coating formulation includes at least one buffer. Examples of suitable buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

In one embodiment of the invention, the coating formulation includes at least one antioxidant, which can comprise a sequestering agent, such sodium citrate, citric acid, EDTA (ethylene-dinitrilo-tetraacetic acid) or a free radical scavenger, such as ascorbic acid, methionine, sodium ascorbate and the like. Presently preferred antioxidants include EDTA and methionine.

In the noted embodiments of the invention, the concentration of the antioxidant is preferably in the range of approximately 0.01-20 wt. % of the coating formulation. More preferably, the concentration of the antioxidant is in the range of approximately 0.03-10 wt. % of the coating formulation.

In one embodiment of the invention, the coating formulation includes at least one surfactant, which can be zwitterionic, amphoteric, cationic, anionic, or nonionic. Suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laurate, and alkoxylated alcohols, such as laureth-4.

In one embodiment of the invention, the concentration of the surfactant is preferably in the range of approximately 0.01-20 wt. % of the coating formulation. More preferably, the concentration of the surfactant is in the range of approximately 0.05-1 wt. % of the coating solution formulation.

In a further embodiment of the invention, the coating formulation includes at least one polymeric material or polymer that has amphiphilic properties, which can include, without limitation, cellulose derivatives, such as hydroxyethylcellulose (HEC), hydroxypropylmethylcellulose (HPMC), hydroxypropylcellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation.

In another embodiment, the coating formulation includes a hydrophilic polymer selected from the following group: hydroxyethyl starch, dextran, poly(vinyl alcohol), poly(ethylene oxide), poly(2-hydroxyethylmethacrylate), poly(n-vinyl pyrolidone), polyethylene glycol and like polymers and mixtures thereof.

In a preferred embodiment, the concentration of the hydrophilic polymer is in the range of approximately 1-30 wt. %, more preferably, in the range of approximately 1-20 wt. % of the coating formulation.

In another embodiment of the invention, the coating formulation includes a biocompatible carrier, which can include, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier is in the range of approximately 2-70 wt. %, more preferably, in the range of approximately 5-50 wt. % of the coating formulation.

In another embodiment, the coating formulation includes a stabilizing agent, which can include, without limitation, a non-reducing sugar, a polysaccharide or a reducing sugar. Suitable non-reducing sugars include, for example, sucrose, trehalose, stachyose, or raffinose. Suitable polysaccharides include, for example, dextran, soluble starch, dextrin, and inulin. Suitable reducing sugars include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides, such as, for example, primeverose, vicianose, rutinose, scillabiose, cellobiose, gentiobiose, lactose, lactulose, maltose, melibiose, sophorose, and turanose, and the like.

Preferably, the concentration of the stabilizing agent is at a ratio of approximately 0.1-2.0%, more preferably, at a ratio of approximately 0.25-1.0% with respect to the biologically active agent.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the coating formulations and, hence, solid biocompatible coatings of the invention is particularly useful to prevent bleeding that can occur following application of the microprojection device or array and to prolong the pharmacokinetics of the active agent through reduction of the blood flow at the application site and reduction of the absorption rate from the skin site into the system circulation.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can include, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilizing/complexing agent, which can include Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred solubilizing/complexing agents are beta-cyclodextrin, hydroxypropyl beta-cyclodextrin, 2-hydroxypropyl-beta-Cyclodextrin and sulfobutylether7 beta-cyclodextrin.

The concentration of the solubilizing/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent comprises in the range of approximately 1 wt. % to 50 wt. % of the coating formulation.

Preferably, the biocompatible coatings formed from coating formulations of the invention have a viscosity less than approximately 500 centipoise and greater than 3 centipoise.

In one embodiment of the invention, the thickness of the biocompatible coating is preferably less than 25 microns, more preferably, less than 10 microns, as measured from the microprojection surface.

### EXAMPLES

The following examples are given to enable those skilled in the art to more clearly understand and practice the present invention. They should not be considered as limiting the scope of the invention but merely as being illustrated as representative thereof.

A method has been devised to calculate the distribution of ionic species in polypeptides and other electrolytes. Equations for equilibrium calculations have been available for many years. They are based on the classic equilibrium laws. They can be used successfully to calculate the net charge of polyelectrolytes such as polypeptides as well as the pI of a protein. Net charge and pI calculations are powerful tools for characterizing and purifying polypeptides. Nevertheless, these calculations do not yield direct information about the species present in solution at a specific pH. For example, the pH range in which species with suspected low solubility are present are not predicted from these methods. Various attempts have been made to estimate the equilibria between different ionic forms in polyelectrolytes. These attempts have been summarized by Edsall J. T. (Proteins as acids and bases, in proteins, amino acids and peptides as ions and dipolar ions, Cohn E. J. & Edsall J. T. eds.; Hafner Pub.; New York and London, 1943, 444-505).

The most successful approach describes a probability distribution function for a system of independently ionizing groups. In this treatment, various groups are classified by classes, each corresponding to one pKₐ value. The procedure is somewhat cumbersome and is not easily amenable to automatic computation. In addition, calculations are limited to the net charged species and do not include description of the charge distribution within the molecule. Surprisingly, with polyelectrolytes, very little attention has been paid to the concentrations of the actual species that are present in solution. This seems to be the result of the lack of equations describing the distribution of species in the presence of overlapping pKₐ values, that is, two or more pKₐ values separated by less than about 3 pH unit. In this case, approximations are being used to calculate the distribution of the species. In a polypeptide molecule, where more than ten overlapping pKₐ values is commonplace, computations based on these approximations are not practical and would certainly yield erroneous results. As a result, distribution of species in polypeptides apparently have not been described. A method has been devised that provides equations describing the species distribution for any polyelectrolyte, provided that their pKₐ values are known. A computational algorithm for performing these calculations is also provided.

### Methods

For polypeptides, the acid-base radicals implicated and their pKₐ values are, respectively: terminal carboxyl, pKₐ=3.05; β-carboxyl of aspartate, pKₐ=3.93; γ-carboxyl of glutamate, pKₐ=4.43; thiol of cysteine, pKₐ=8.38; phenyl of tyrosine, pKₐ=10.36; imidazolium of histidine, pKₐ=5.96; terminal ammonium, pKₐ=8.1; ε-ammonium of lysine, pKₐ=10.59; guanidinium of arginine, pKₐ=12.48. The above pKₐ values are averages compiled from the literature and used in the examples. The pI values were extrapolated from the net charge profile of the molecule calculated from their pKₐ values.

Determination of the Specie Concentrations in a Polyelectrolyte:

For a weak acid, AH, the equilibrium can be written:
AH [symbol] A⁻+H⁺

Accordingly, the dissociation constant of AH can be represented as:
Kₐ=(A⁻)×(H⁺)/(AH), where
(A⁻), (H⁺), and (AH) are the concentrations of the species.

From this equation, the classic Henderson-Hasselbalch equation can be derived:
pH=pKₐ+Log((A⁻)/(AH))

Assuming that: (A⁻)+(AH)=1, this equation yields:
Mole fraction neutral=1/(1+10^{pH-pKa})=P
   which can also be defined as the probability of the acid to be neutral.

The same equation also indicates the probability that the acid is ionized:
Mole fraction ionized, negatively charged=1−1/(1+10^{pH-pKa})=1−P
Net charge=1/(1+10^{pH-pKa})−1

As is known in the art, similar equations can be derived for a base. Specifically, for a weak base, B, the equilibrium can be written:
B+H⁺ [symbol] BH⁺

Accordingly, the dissociation constant of BH can be represented as:
Kₐ=(B)×(H⁺)/(BH⁺), where
(B), (H⁺), and (BH⁺) are the concentrations of the species.

As above, the Henderson-Hasselbalch equations can be derived:
pH=pKₐ−Log (BH⁺/B),
Mole fraction neutral=1/(1+10^{pKa-pH})=Q
Mole fraction ionized, positively charged=1−1/(1+10^{pKa-pH})=1−Q
Net charge=1−1/(1+10^{pKa-pH})

The species are defined as all the possible combinations of the charges for the acidic functions and basic functions of the compound in solution. For example, if the compound presents only acidic functions, the species take the values like 0⁻, 1⁻, 2⁻, and etc. Similarly, if the compound presents only basic functions, the species take the values like 0⁺, 1⁺, 2⁺, and etc. If the compound has both acidic and basic functions, then the species take the values of 0⁻ 0⁺, 0⁻ 1⁺, 1⁻ 0⁺, 1⁻ 1⁺, etc. The net charged species are defined as the sum of all species presenting an identical net charge. For example, the net charges take the values: . . . −2, −1, 0, +1, +2 . . .

In a first example, for a compound bearing one acidic (negatively charged) pKₐ, the species present in solution at any pH are 0⁻ and 1⁻ (one species is neutral: no positive charge and no negative charge; the other species has one negative charge and no negative charge).

Thus, the mole fraction of these species at a specific pH is:

0⁻: P₁
1⁻: 1−P₁
where P₁ is the probability of the acidic group being neutral.

In another example, for a compound bearing one acidic pKₐ, and one basic (positively charged) pKₐ, the species present in solution at a specific pH are: 0⁻ 0⁺, 0⁻ 1⁺, 1⁻ 0⁺, 1⁻ 1⁺. Thus, the mole fraction of these species at a specific pH is:
0⁻ 0⁺: P₁×Q₁
0⁻ 1⁺: P₁×(1−Q₁)
1⁻ 0⁺: (1−P₁)×Q₁
1⁻ 1⁺: (1−P₁)×(1−P₁)
where P₁ and Q₁ are the probability of the acidic and basic group, respectively, being neutral.

In yet another example, for a compound bearing one acidic pKₐ, and two basic pKₐ, the species present in solution at any pH are: 0⁻ 0⁺, 0⁻ 1⁺, 0⁻ 2⁺, 1⁻ 0⁺, 1⁻ 1⁺, 1⁻ 2⁺. Thus, the mole fraction of these species at a specific pH is:
0⁻ 0⁺: P₁×Q₁×Q₂
0⁻ 1⁺: (P₁×Q₁×(1−Q₂))+(P₁×(1−Q₁)×Q₂)
0⁻ 2⁺: P₁×(1−Q₁)×(1−Q₂)
1⁻ 0⁺: (1−P₁)×Q₁×Q₂
1⁻ 1⁺: ((1−P₁)×Q₁×(1−Q₂))+((1−P₁)×(1−Q₁)×Q₂)
1⁻ 2⁺: (1−P₁)×(1−Q₁)×(1−Q₂)
Etc . . .
where P₁, Q₁ and Q₂ are the probability of the acid and basic groups, respectively, being neutral.

The above examples demonstrate that there are (N+1) (M+1) species, where N and M are the number of acidic and basic pKₐs, respectively. In the previous example, there were six possible species. Thus, the number of possible net charged species is (N+M+1). As demonstrated above, the mole fraction of the net charged species at a specific pH can be deduced. Using the preceding example, the probabilities for the possible net charged species are:

$$\begin{matrix}- 1 \text{:} ( 1 - {P}_{1} ) \times {Q}_{1} \times {Q}_{2} \\ 0 \text{:} ( {P}_{1} \times {Q}_{1} \times {Q}_{2} ) + ( ( 1 - {P}_{1} ) \times {Q}_{1} \times ( 1 - {Q}_{2} ) ) + \\ ( ( 1 - {P}_{1} ) \times ( 1 - {Q}_{1} ) \times {Q}_{2} ) \\ + 1 \text{:} ( {P}_{1} \times {Q}_{1} \times ( 1 - {Q}_{2} ) ) + ( {P}_{1} \times ( 1 - {Q}_{1} ) \times {Q}_{2} ) + \\ ( 1 - {P}_{1} ) \times \times ( 1 - {Q}_{1} ) \times ( 1 - {Q}_{2} ) \\ + 2 \text{:} {P}_{1} \times ( 1 - {Q}_{1} ) \times ( 1 - {Q}_{2} )\end{matrix}$$

Computational Algorithm of the Species and Valences of a Polyelectrolyte:

Based on the above equations, an algorithm has been derived which is used to calculate the charge, net charge, species and valences present in a polyelectrolyte. In the following examples, a bold and an upper case letter is used to denote a vector or a matrix, and a lower case letter is used to represent an element of the vector or the matrix.

Generally, a given compound has N acidic functions and M basic functions, given pKₐ for the functions, and is present in a solution of a given pH. PKAₐ can e defined as the N by 1 vector of acidic pKₐ values, and PKA_{b} as the M by 1 vector of basic pKₐ values:
PKAₐ=(pKaₐ₁, pKaₐ₂, . . . , pKa_{aN})^{T}
PKA_{b}=(pKa_{b1}, pKa_{b2}, . . . , pKa_{bM})^{T}
P═(p₁, p₂, . . . , p_{N})^{T}
Q=(q₁, q₂, . . . , q_{M})^{T}
pᵢ=/(1+10^{pH-pKa}^{ₐᵢ})
qⱼ=1/(1+10^{pKa}^{_{bi}}^{-pH})

In the above equation, P and Q represent the mole fraction neutral for acidic components and basic functions, respectively. As discussed above, P and Q also represent the probabilities of being neutral for either acid or base.

Further, CHARGEₐ can denote the N by 1 vector of charge for the acids, while CHARGE_{b} can denote the M by 1 vector for the bases, as follows:
CHARGEₐ=(chargeₐ₁, chargeₐ₂, . . . , charge_{aN})^{T}
CHARGE_{b}=(charge_{b1}, charge_{b2}, . . . , charge_{bM})^{T}

$$\begin{matrix}{charge}_{ai} = 1 / ( 1 + {10}^{p H - {pKa}_{ai}} ) - 1 \\ {charge}_{bj} = 1 - 1 / ( 1 + {10}^{{pKa}_{bj} - p H} ) \\ net charge = {\sum }_{i = 1}^{N} {charge}_{ai} + {\sum }_{j = 1}^{M} {charge}_{bj} ,\end{matrix}$$
where net charge is the charge of the complex molecule in the solution.

Again, given the generalized compound discussed above, the species of the compound can also be determined. For simplicity, a is used to denote the species. In a first example, if a compound only has N acids, the probabilities of a in the solution can be derived. As can be determined from the equations above, P is the probability vector for the acids being neutral. In one situation, a solution can be made by adding one acid and one acid. At the beginning, when only one acid is in the solution, the probabilities are:
Prob(α=0⁻, 1 acid)=p₁
Prob(α=1⁻, 1 acid)=1−p₁
Prob(α=2⁻, 1 acid)= . . . =Prob(α=N⁻, 1 acid)=0

Further, given that i acids are already in the solution, the probabilities can be determined for the addition of one more. Accordingly, the relationships of the probabilities are:
Prob(α=0⁻, i+1 acids)=Prob(α=0⁻, i acids | the (i+1)th acid=0) Prob(the i+1 th acid=0)
Prob(α=j⁻, i+1 acids)=Prob(α=j⁻, i acids | the (i+1)th acid=0) Prob(the i+1 th acid=0)+Prob(α=(j−1)−, i acids | the (i+1)th acid=1) Prob(the i+1 th acid=1)

Given an assumption that all the acids are independent, the above equations can be represneted as follows:
Prob(α=0⁻, i+1 acids)=Prob(α=0⁻, i acids) Prob(the i+1 th acid=0)
Prob(α=j⁻, i+1 acids)=Prob(α=j⁻, i acids) Prob(the i+1 th acid=0)+Prob(α=(j−1)−, i acids) Prob(the i+1 th acid=1)

As can be appreciated, the above equations represtent an useful way to calculate the probabilities. To implement them, R can designate a N+1 by N matrix:
r[j,i]=Prob(α=(j−1)⁻, i acids)

Given this designation, the above equations can be rewritten as:
r[1,1]=P₁
r[2,1]=1−P₁
r[3,1]= . . . =r[N+1,1]=0
r[1,i+1]=r[1,i] pᵢ₊₁
r[j+1, i+1]=r[j+1, i] pᵢ₊₁+r[j,i](1−pᵢ₊₁),
where i=1 . . . (N−1) and j=1, . . . , N

As will be appreciated, the above recursion algorithm by loops can be coded, and the last column of R simply represents the probabilities of species when a compound with N acids is in the solution. Given the same general conditions, A can represent the last column of R and B can represent the species probability vector when a compound of M bases is in the solution, and the dimension is M+1 by 1. The determination of B can be obtained in the same manner as A. Thus, if the compound has N acids and M bases, the probabilities of species are:
C=A×B^{T}
c[i,j]=Prob(α=(i−1)⁻(j−1)⁺),
where I=1, 2, . . . , N+1 and j=1, 2, . . . , M+1, and
where C is an N+1 by M+1 matrix.

At last, the net charged species (β) can be constructed based on C:

$$\begin{matrix}Prob ( β = i ) = {\sum }_{{i = k - j }_{\begin{matrix}k = 1 , … , M + 1 \\ j = 1 , … , N + 1 \end{matrix}}} c [ j , k ] \\ where i = - N , … , - 1 , 0 , 1 , … , M\end{matrix}$$

By applying the general concepts and equations discussed above, the distribution of charged or neutral species for selected compounds can be calculated. The following examples illustrate such determinations.

### Example 1

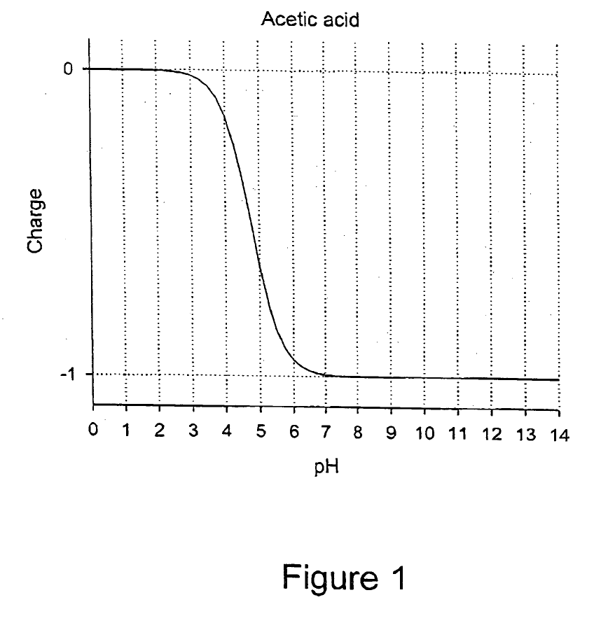

FIG. 1 shows the charge profile of acetic acid (pKₐ 4.75) as a function of pH. At pH below about 2.5 the carboxyl group of the acetic acid is completely protonated and thus there is no charge on the molecule. As the pH increases from about 2.5 to about 7, more and more of the carboxyl moieties become ionized and thus forming the negatively charged acetate ion. At about pH 7, all of the carboxyl groups are ionized.

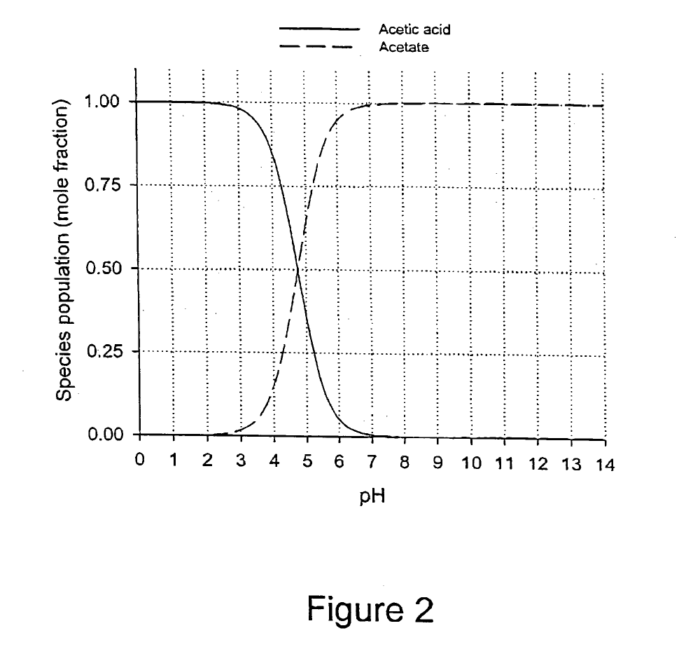

FIG. 2 shows the mole ratios of acetic acid and acetate. At pH 0, with the carboxyl group of acetic acid fully protonated, there is essentially only acetic acid, thus the mole fraction is 1. At about pH 2.5, ionization of the carboxyl group begins and the solid curve representing acetic acid in graph starts to move downward. At the same time, the dashed line, representing the ionized acetate, starts to move upwards off of the 0.00 line. At about pH 4.7 there are equal numbers of charged and uncharged moieties. At pH greater than about 7, there is no longer any uncharged acetic acid and essentially all of species are the charged acetate ion.

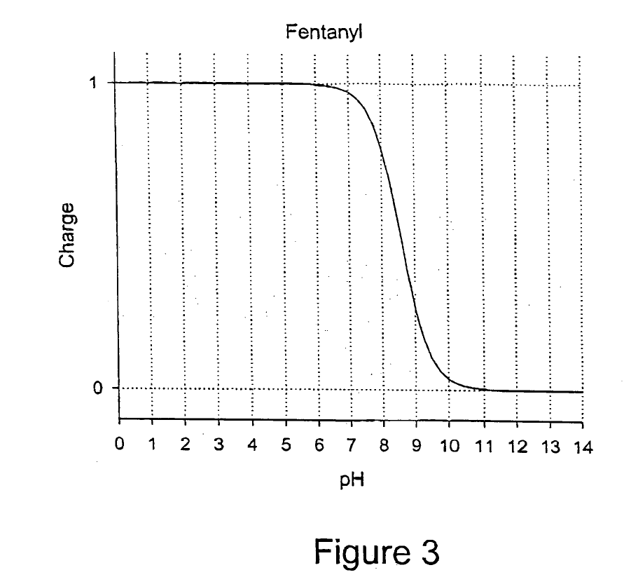

Many agents exhibit maximum solubility in water when they are in an electrically charged state. FIG. 3 shows the charge profile of fentanyl, a small molecular weight weakly basic agent presenting one basic pKₐ, 8.5. At pH below 6, essentially all of the fentanyl is positively charged, while at pH above 11, essentially all of the fentanyl is neutral.

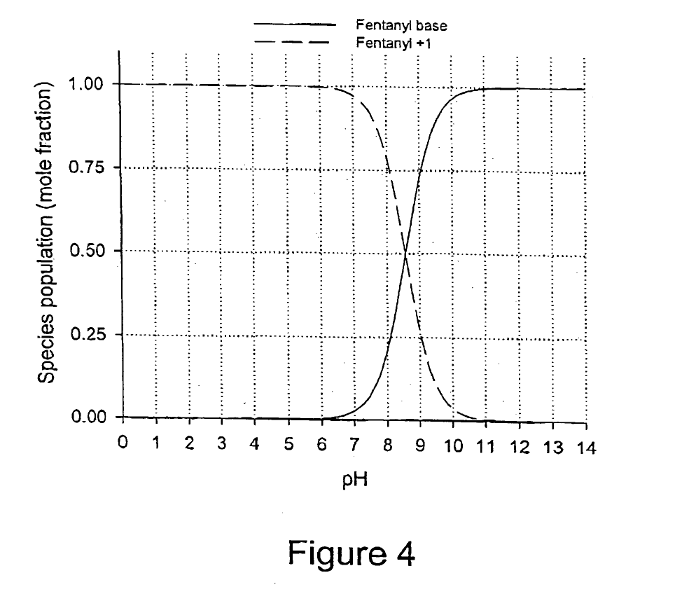

FIG. 4 shows the mole ratios of the neutral (fentanyl base-solid line) and charged fentanyl (fentanyl⁺¹—dashed line) species at different pHs. From pH 0 to about pH 6, there is essentially no fentanyl base present and 100% is the charged fentanyl⁺¹. From pH about 6 to about pH 11, there is a transition. The fentanyl⁺¹ decreases at the same rate that the fentanyl base increases. At or above pH 11, essentially all of the fentanyl exists in the non-charged, neutral, fentanyl base.

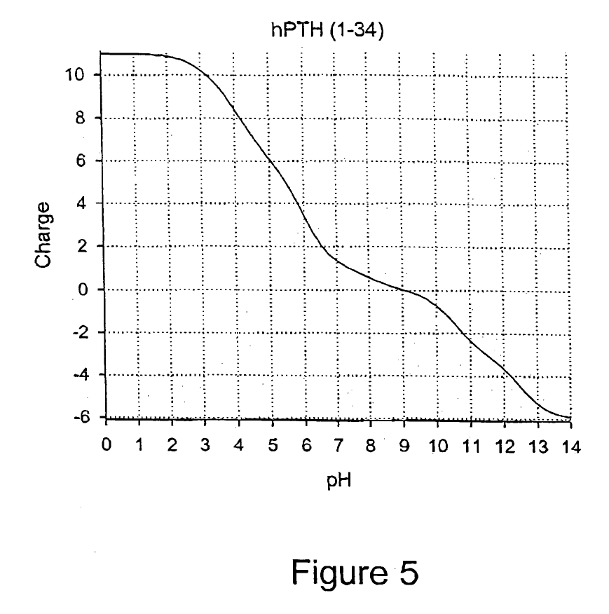

Complex molecules such as peptides and proteins also exhibit charge characteristics that are dependant on pH. FIG. 5 shows the charge profile of hPTH(1-34)OH, a peptide presenting 11 basic pKₐ's, and six acidic pKa's. At pH 9, the peptide presents a zero net electric charge. This point is also called the isoelectric point or pI.

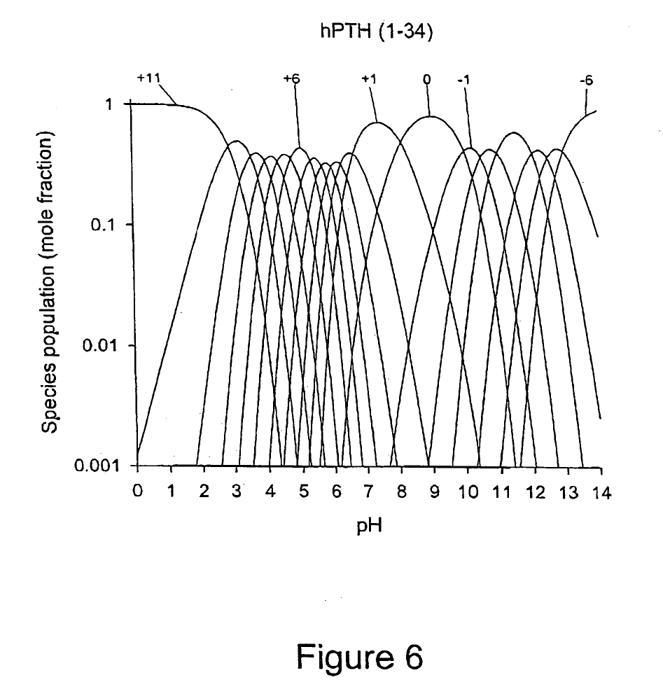

FIG. 6 shows the mole ratios of the net charged species of PTH. The species range from a +11 charge to a −6 charge. The neutral species only exist in significant amounts in the pH range of about 6 to about 11.5. In this pH range, PTH precipitates out of solution.

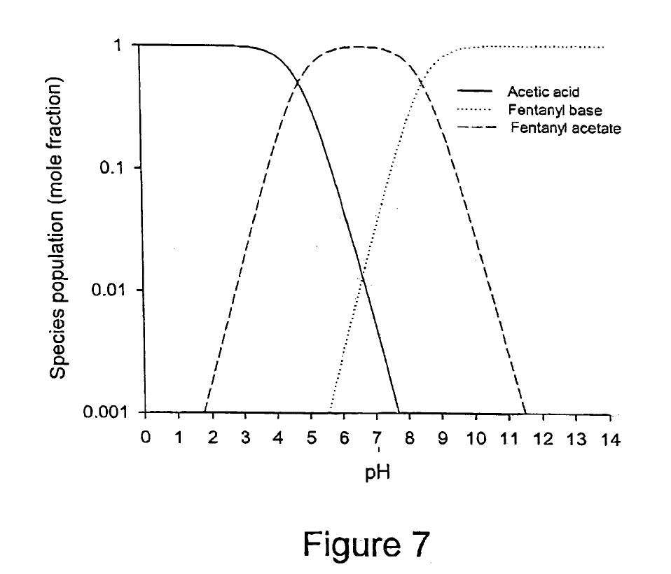

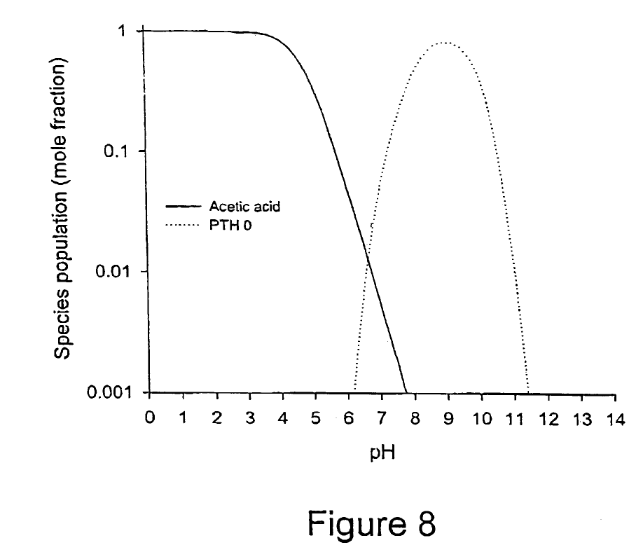

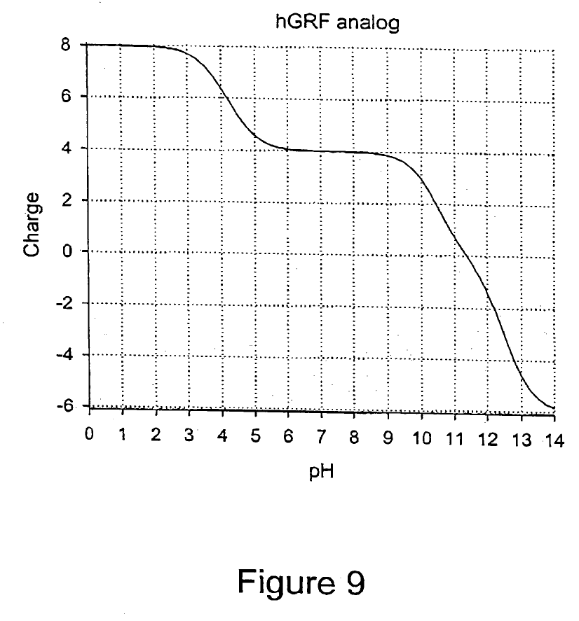

FIG. 7 shows the mole ratios for fentanyl acetate (dashed line), acetic acid (solid line), and the neutral form of fentanyl (fentanyl base-dotted line). These are the species that are present in solution at different pH's when various ratios of fentanyl base and acetic acid are mixed in solution. The pH of fentanyl acetate (mole ratio 1 to 1) in solution is predicted to be about 6.6. At that pH, about 1% of fentanyl is present as fentanyl base, which, for a 10 mg/mL solution total fentanyl, would be at or above the limit of solubility of the base, which would therefore precipitate out. Solubilization can be achieved by supplementing the formulation with excess acetic acid, which will result in acidification of the formulation and reduction of the amount of fentanyl base. Nevertheless, during drying and subsequent storage the free acetic acid will evaporate which will ineluctably result in the formation of the water insoluble base. Subsequent reconstitution in water would not allow rapid solubilization of fentanyl.

The use of a mixture of non-volatile counterion and volatile counterions would provide a water soluble formulation, that upon drying would lose, at least partially, its volatile counterion content, therefore reconstituting the base. Upon exposure to biological fluids, the non-volatile and volatile counterions associated with fentanyl would provide a rapidly soluble form of fentanyl, while the base form of fentanyl would be slowly soluble in the biological fluids, thereby providing a sustained release formulation.

### Example 2

160 mg fentanyl hydrochloride and 40 mg fentanyl acetate are solubilized in 10 mL water. The coating solution is then applied to the microprojections using the coating methods described in U.S. Publication No. 2002/0132054. The coating is analyzed for fentanyl content and is found to contain 80% fentanyl hydrochloride, 5% fentanyl acetate, and 15% fentanyl base (expressed as mole %). When the device is applied in humans using the applicator described in U.S. Publication 2002/0123675, fast onset is observed followed by sustained delivery of fentanyl.

### Example 3

100 mg fentanyl hydrochloride and 100 mg fentanyl base are solubilized in 10 mL ethanol. The coating solution is then applied to the microprojections using the coating methods described in U.S. Publication No. 2002/0132054. The coating is analyzed for fentanyl content and is found to contain 50% fentanyl hydrochloride, and 50% fentanyl base. When the device is applied in humans using the applicator described in U.S. Publication 2002/0123675, sustained delivery of fentanyl is achieved following a rapid onset.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A composition for coating a transdermal delivery device having stratum corneum-piercing microprojections comprising a formulation of a biologically active agent, a non-volatile counterion and a volatile counterion, wherein said non-volatile counterion causes the formation of a first species of said biologically active agent that has improved solubility when said formulation is dried and wherein said volatile counterion causes the formation of a second species of said biologically active agent that has reduced solubility when said formulation is dried.

2. The composition of claim 1, wherein said first species is adapted to rapidly provide a therapeutically relevant blood level of said biologically active agent when said formulation is allowed to dissolve in a bodily fluid.

3. The composition of claim 1, wherein said second species is adapted to provide a sustained therapeutically relevant blood level of said biologically active agent when said formulation is allowed to dissolve in a bodily fluid.

4. The composition of claim 1, comprising approximately equimolar amounts of said non-volatile counterion and said volatile counterion.

5. The composition of claim 1, wherein said formulation has a pH, said biologically active agent has a positive charge at said formulation pH and said non-volatile counterion comprises a non-volatile weak acid.

6. The composition of claim 6, wherein said non-volatile weak acid has an acidic pKa and a property selected from the group consisting of a melting point higher than about 50° C. and a boiling point higher than about 170° C. at atmospheric pressure.

7. The composition of claim 6, wherein said non-volatile weak acid is selected from the group consisting of citric acid, succinic acid, glycolic acid, gluconic acid, glucuronic acid, lactic acid, malic acid, pyruvic acid, tartaric acid, tartronic acid, and fumaric acid.

8. The composition of claim 1, wherein said formulation has a pH, said biologically active agent has a positive charge at said formulation pH and said non-volatile counterion comprises a strong acid.

9. The composition of claim 8, wherein said strong acid has at least one pKa lower than about 2.

10. The composition of claim 9, wherein said strong acid is selected from the group consisting of hydrochloric acid, hydrobromic acid, nitric acid, sulfonic acid, sulfuric acid, maleic acid, phosphoric acid, benzene sulfonic acid and methane sulfonic acid.

11. The composition of claim 1, wherein said formulation has a pH, said biologically active agent has a positive charge at said formulation pH and said non-volatile counterion comprises an acidic zwitterion.

12. The composition of claim 11, wherein said acidic zwitterion has at least two acidic pKas and at least one basic pKa, so that there is at least one acidic pKa more than said basic pKas.

13. The composition of claim 12, wherein said acidic zwitterion is selected from the group consisting of glutamic acid and aspartic acid.

14. The composition of claim 1, wherein said formulation has a pH, said biologically active agent has a negative charge at said formulation pH and said non-volatile counterion comprises a non-volatile weak base.

15. The composition of claim 14, wherein said non-volatile weak base has a basic pKa and a property selected from the group consisting of a melting point higher than about 50° C. and a boiling point higher than about 170° C. at atmospheric pressure.

16. The composition of claim 15, wherein said non-volatile weak base is selected from the group consisting of monoethanolomine, diethanolamine, triethanolamine, tromethamine, methylglucamine, glucosamine.

17. The composition of claim 1, wherein said formulation has a pH, said biologically active agent has a negative charge at said formulation pH and said non-volatile counterion comprises a strong base.

18. The composition of claim 17, wherein said strong base has at least one pKa higher than about 12.

19. The composition of claim 18, wherein said strong base is selected from the group consisting of sodium hydroxide, potassium hydroxide, calcium hydroxide, and magnesium hydroxide.

20. The composition of claim 1, wherein said formulation has a pH, said biologically active agent has a negative charge at said formulation pH and said non-volatile counterion comprises a basic zwitterion.

21. The composition of claim 20, wherein said basic zwitterion has at least two basic pKas and at least one acidic pKa, so that there is at least one basic pKa more than acidic pKas.

22. The composition of claim 21, wherein said basic zwitterion is selected from the group consisting of histidine, lysine, and arginine.

23. The composition of claim 1, wherein said formulation has a pH, said biologically active agent has a positive charge at said formulation pH and said non-volatile counterion comprises a mixture of counterions comprising at least one non-volatile strong acid and at least one non-volatile weak acid.

24. The composition of claim 1, wherein said formulation has a pH, said biologically active agent has a negative charge at said formulation pH and said non-volatile counterion comprises a mixture of counterions comprising at least one non-volatile strong base and at least one non-volatile weak base.

25. The composition of claim 1, wherein said formulation has a pH, said biologically active agent has a positive charge at said formulation pH and said volatile counterion comprises a volatile weak acid.

26. The composition of claim 25, wherein said volatile weak acid has an acidic pKa higher than approximately 2 and a property selected from the group consisting of a melting point lower than about 50° C. and a boiling point lower than about 170° C. at Pₐₜₘ.

27. The composition of claim 26, wherein said volatile weak acid is selected from the group consisting of acetic acid, propionic acid and pentanoic acid.

28. The composition of claim 1, wherein said formulation has a pH, said biologically active agent has a negative charge at said formulation pH and said volatile counterion comprises a volatile weak base.

29. The composition of claim 28, wherein said volatile weak acid has a basic pKa lower than approximately 12 and a property selected from the group consisting of a melting point lower than about 50° C. and a boiling point lower than about 170° C. at Pₐₜₘ.

30. The composition of claim 29, wherein said volatile weak base is selected from the group consisting of ammonia and morpholine.

31. The composition of claim 1, wherein said biologically active agent is selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

32. The composition of claim 1, wherein said biologically active agent comprises a fentanyl-based agent selected from the group consisting of fentanyl, fentanyl base, fentanyl salt, alpha-methyl fentanyl, 3-methyl fentanyl, methyl fentanyl, remifentanyl, sufentanyl, alfentanyl, lofentanyl and carfentanyl.

33. The composition of claim 32, wherein said fentanyl salt is formed in conjunction with an ion selected from the group consisting of acetate, propionate, butyrate, pentanoate, hexanoate, heptanoate, levulinate, chloride, bromide, citrate, succinate, maleate, glycolate gluconate, glucuronate, 3-hydroxyisobutrate, 2-hydroxyisobutyrate, lactate, malate, pyruvate, fumarate, tartarate, tartronate, nitrte, phosphate, benzene sulfonate, methane sulfonate, sulfate, sulfonate, tricarballylicate, malonate, adipate, citraconate, glutarate, itaconate, mesaconate, citramalate, dimethylolpropionate, tiglicate, glycerate, methacrylate, isocrotonate, b-hydroxybutyrate, crotonate, angelate, hydracrylate, ascorbate, aspartate and glutamate.

34. The composition of claim 31, wherein said formulation includes said fentanyl-based agent in the range of approximately 1-60 wt. % of said formulation.

35. The composition of claim 34, wherein said formulation includes said fentanyl-based agent in the range of approximately 5-30 wt. % of said formulation.

36. The composition of claim 31, wherein said formulation has a pH in the range of approximately 1-6.

37. The composition of claim 36, wherein said formulation has a pH in the range of approximately 2-5.5.

38. The composition of claim 1, wherein said formulation further comprises a formulation adjuvant.

39. The composition of claim 38, wherein said formulation adjuvant comprises a buffer.

40. The composition of claim 38, wherein said formulation adjuvant comprises an antioxidant.

41. The composition of claim 38, wherein said formulation adjuvant comprises a surfactant.

42. The composition of claim 38, wherein said formulation adjuvant comprises an amphiphilic polymer.

43. The composition of claim 38, wherein said formulation adjuvant comprises a hydrophilic polymer.

44. The composition of claim 38, wherein said formulation adjuvant comprises a biocompatible carrier.

45. The composition of claim 38, wherein said formulation adjuvant comprises a stabilizing agent.

46. The composition of claim 38, wherein said formulation adjuvant comprises a vasoconstrictor.

47. The composition of claim 38, wherein said formulation adjuvant comprises a pathway patency modulator.

48. The composition of claim 38, wherein said formulation adjuvant comprises a solubilising/complexing agent.

49. The composition of claim 38, wherein said formulation adjuvant comprises a non-aqueous solvent.

50. The composition of claim 1, wherein said formulation has a viscosity less than about 500 centipoise and greater than about 3 centipoise.

51. The composition of claim 1, further comprising a transdermal delivery device having at least one microprojection configured to pierce the stratum corneum, wherein said formulation is coated on said microprojection and dried.

52. The device of claim 51, wherein said formulation coated on said microprojection has a thickness less than approximately 25 microns.

53. The device of claim 52, wherein said formulation coated on said microprojection has a thickness less than approximately 10 microns.

54. A method for transdermally delivering a biologically active agent comprising the steps of:
providing a transdermal delivery device having at least one stratum corneum-piercing microprojection, the microprojection including a biocompatible coating comprising a dried formulation of said biologically active agent, a non-volatile counterion and a volatile counterion, wherein said non-volatile counterion causes the formation of a first species of the biologically active agent that has improved solubility when said formulation is dried and said volatile counterion causes the formation of a second species of said biologically active agent that has reduced solubility when said formulation is dried; and
applying said delivery device to a patient to deliver said biologically active agent.

55. The method of claim 54, further comprising the step of rapidly establishing a therapeutically relevant blood level of said agent in said patient by dissolving said first species.

56. The method of claim 55, wherein the step of rapidly establishing a therapeutically relevant blood level of said agent comprises establishing said blood level in less than 30 min after applying said device.

57. The method of claim 56, wherein the step of rapidly establishing a therapeutically relevant blood level of said agent comprises establishing said blood level in less than 15 min after applying said device.

58. The method of claim 55, further comprising the step of maintaining a therapeutically relevant blood level of said agent in said patient by dissolving said second species.

59. The method of claim 58, wherein the step of maintaining a therapeutically relevant blood level of said agent comprises maintaining said blood level in the range of approximately 1 to 6 hours.

60. The method of claim 59, wherein the step of maintaining a therapeutically relevant blood level of said agent comprises maintaining said blood level in the range of approximately 2 to 4 hours.

61. The method of claim 54, wherein said agent comprises a fentanyl-based agent.

62. The method of claim 58, wherein said agent comprises fentanyl and wherein said therapeutically relevant blood level is at least approximately 0.3 ng/mL.

63. The method of claim 62, further comprising the step of delivering said agent in the range of approximately 10 to 1000 mg per day.

64. A method for applying a biocompatible coating to a transdermal delivery device that has a least one stratum corneum-piercing microprojection comprising the steps of:
providing a formulation of a biologically active agent, a non-volatile counterion, and a volatile counterion, wherein said non-volatile counterion causes the formation of a first species of said biologically active agent that has improved solubility when the formulation is dried and wherein said volatile counterion causes the formation of a second species of said biologically active agent that has reduced solubility when said formulation is dried;
applying said formulation to said microprojection; and
drying said formulation.
